(19) United States
(12) Patent Application Publication
(10) Pub. No.: US 2012/0046228 A1
(43) Pub. Date: Feb. 23, 2012

(54) Methods for Modulating Ovulation

(75) Inventor: Carlos A. Molina, Bloomfield, NJ (US)

(73) Assignee: UNIVERSITY OF MEDICINE AND DENTISTRY OF NEW JERSEY, Somerset, NJ (US)

(21) Appl. No.: 13/212,682

(22) Filed: Aug. 18, 2011

Related U.S. Application Data
(60) Provisional application No. 61/374,863, filed on Aug. 18, 2010.

(51) Int. Cl. A61K 38/17; A61P 15/00; A61K 38/24; A61P 15/08; A61K 38/02; A61K 31/7088

(52) U.S. Cl. 514/9.9; 514/9.8; 514/44 R; 514/10.1; 514/1.1; 514/44 A

(57) ABSTRACT

The present invention relates to methods and reagents for modulating ovulation in humans and non-human animals.

### CROSS REFERENCE TO RELATED APPLICATION

This application claims priority of U.S. Provisional Application No. 61/374,863, filed on Aug. 18, 2010. The content of the application is incorporated herein by reference in its entirety.

### GOVERNMENT INTERESTS

The invention disclosed herein was made, at least in part, with Government support under Grant Nos R03HD045503 and F31HD43691 from the National Institute of Child Health and Human Development/National Institutes of Health. Accordingly, the U.S. Government has certain rights in this invention.

### FIELD OF THE INVENTION

This invention relates to reagents and methods for modulating ovulation.

### BACKGROUND OF THE INVENTION

Ovulation is the process by which an ovum or ova are released from the ovaries. A fundamental prerequisite for achieving successful reproduction, it is a complex biological response controlled by the cyclical action of hormones, particularly the gonadotropins FSH and LH. Many people can benefit from modulating ovulation in both humans and non-human animals. For example, in humans, increased rates of infertility accompanied by the delay in age at marriage and declining birthrates have been global problems in advanced countries. According to American Society for Reproductive Medicine and Centers for Disease Control and Prevention, about 10% of women (6.1 million) in the United States ages 15-44 years have infertility problems. Treatment of infertility often requires ovulation induction and/or controlled ovarian hyperstimulation. On the other hand, contraception can be achieved by suppressing ovulation. In non-human animals, productivity of a livestock herd, particularly meat or egg producing livestock, depends largely on the reproductive efficiency of the herd.

Hormonal stimulation has been used in modulating ovulation. However, it has various drawbacks including risks of ovarian hyper stimulation syndrome, weight gain, bloating, nausea, vomiting, and long-term risks of cancer. Thus, there still is a need for new reagents and methods for modulating ovulation in both humans and non-human animals.

### SUMMARY OF INVENTION

This invention relates to agents and methods for modulating ovulation in both humans and non-human animals.

In one aspect, this invention features a method of increasing ovulation in a vertebrate animal having an ovary. The method includes a step of administering to the animal a first composition containing (i) an inducible cAMP early repressor (ICER) polypeptide or (ii) a vector having a nucleic acid sequence encoding the polypeptide. Examples of the ICER polypeptide include mouse/rat and human ICER polypeptides i.e., those having the sequences of SEQ ID NOs: 1-8 listed below. In one embodiment, the method includes another step of administering to the animal a second composition that stimulates ovulation. The method can further include a step of collecting or harvesting one or more ova from the animal. The ova collected can be used in in vitro fertilisation (IVF) and related procedures.

The second composition can contain a hormonal or a chemical stimulant of ovulation, such as a gonadotropin hormone selected from the group consisting of pregnant mare serum gonadotropin (PMSG), human menopausal gonadotropin (hMG), follicle stimulating hormone (FSH), luteinizing hormone (LH), human chorionic gonadotropin (hCG), and anti-Müllerian hormone (AMH). The first or second composition can be a pharmaceutical composition. Each can be administered directly to the ovary of the animal.

The aforementioned animal can be a mammal, such as a human. In that case, the method can be used to treat a human that has an infertility disorder, e.g., polycystic ovary syndrome (PCOS) or oligomenorrhea. The aforementioned animal can also be a non-human animal, including a fish or a bird. In one example, the animal is a domestic animal, such as a livestock animal or a pet animal. In another example, the animal is a wild animal or an animal of an endangered species.

In a second aspect, the invention features a kit for treating infertility. The kit includes, among others, the above-mentioned first composition having (i) an inducible cAMP early repressor polypeptide or (ii) a vector comprising a nucleic acid sequence encoding the polypeptide, and the above-mentioned second composition for treating infertility. The second composition can contain a hormonal or a chemical stimulant of ovulation, including those listed above.

In a third aspect, the invention features a method of decreasing ovulation in a vertebrate animal having an ovary. The method includes a step of administering to the animal a composition containing an antagonist (e.g., a protein, a peptide, a small molecule compound, an antisense nucleic acid, or an RNAi agent) of an inducible cAMP early repressor polypeptide. As mentioned above, suppressing ovulation can be used to achieve contraception. Decreasing ovulation therefore can be used in regulating or controlling the rate and timing of pregnancy and birth in humans or non-human animals, e.g., a livestock herd.

The details of one or more embodiments of the invention are set forth in the description below. Other features, objects, and advantages of the invention will be apparent from the description and from the claims.

### BRIEF DESCRIPTION OF THE DRAWINGS

FIGS. 1A-C are (A) a diagram showing a linear representation of the transgene digested with BamH I and Xba I resulting in a 2.18 kb fragment, the line below “*” representing the area used to generate the FLAG-ICER probe; (B) a diagram showing a sequence alignment between the FLAG-ICER cDNA against the CREM gene, with the boxed region representing the location of the ICER-IIγ exons with the 18.5 kb portion of the of the CREM gene, where labeled are the predicted fragments that hybridize with FLAG-ICER probe; and (C) an autoradiograph showing Southern Blot analysis on genomic DNA extracted from F0 pups, where labeled are the ID numbers of positive transgenic founder mice.

FIGS. 2A-D are photographs and a diagram showing characterization of ovarian specific ICER transgenic mice: (A) results of immunoblot analysis of transgene expression in different tissue lysates from mature mice probed with either anti-Flag M2 or anti-ICER antibodies; (B) schematic representation of the 3′ end of the CREM depicting the exons coding for DNA Binding Domains I and II (DBD I and DBD II); (C) results of ribonuclease protection analysis of FLAG-ICER mRNA during exogenous gonadotropin treatment in immature transgenic and wild-type female mice, where, for loading control, β-actin mRNA levels were determined; and (D) results of immunoblot analysis of protein fraction from samples in B probed with either anti-Flag M2 or anti-ICER antibodies.

FIGS. 3A and B are photographs showing granulosa cell specific localization of ICER exogenous expression in transgenic mice, where ovary tissue sections from transgenic mice or wild-type littermate primed with PMSG for 48 hrs followed with hCG treatment for an additional 24 hrs and analyzed by immunohistochemistry using (A) anti-ICER antibody or (B) anti-FLAG M2 antibody; arrows indicate regions with brown staining of FLAG-ICER expression; left hand panels represent 40× magnification of tissue sections; on the right hand panels 100× magnification of tissue section and the location of the area of detail are indicated.

FIGS. 4A and B are diagrams showing hyperovulation and high progesterone levels in immature transgenic mice: (A) number of released oocytes from superovulated transgenic or wild type mice and (B) mean concentration of progesterone in blood serum from superovulated wild-type or transgenic mice, where error bars are s.e.m. and P values were calculated using a one-tailed Student's t-test.

FIGS. 5A and B are diagrams showing high ovulation rate in mature transgenic mice: (A) mean number of released oocytes from mature two-month old transgenic or wild type mice and (B) mean weight of ovaries after ovulation, where error bars indicate s.e.m. and P values were calculated using a one-tailed Student's t-test.

FIGS. 6A-C are diagrams showing levels of FSH, LH, and estrogen, respectively, in transgenic mice and wild type mice.

### DETAILED DESCRIPTION OF THE INVENTION

This invention is based, at least in part, on unexpected discoveries that ovulation, a complex biological process controlled by the cyclical action of hormones, can be modulated by a group of inducible cAMP early repressor (ICER) polypeptides.

The ovary is a dynamic organ undergoing constant change, serving as the site of gamete (oocyte) production as well as reproductive hormone production and secretion. The ovarian follicle fosters the oocyte in addition to functioning as the major endocrine and reproductive compartment of the ovary. Maturation of the individual follicles requires both proliferation and differentiation of the follicular cell compartment. This balance between cellular proliferation and differentiation requires the coordinated expression of specific genes, dependent on the temporal exchange of many extracellular signals such as hormones and growth factors. Granulosa cells, the cells of the follicles, use an array of signaling pathways to interpret these external cues to ultimately control the switching on and off of genes at the appropriate time during follicular maturation. Surges in FSH and estrogen levels stimulate granulosa cells to proliferate, whereas surges in LH levels inhibit cell growth and induce the differentiation into luteal cells. Although it is was known that cAMP signaling plays a pivotal role in gonadotropin regulation of granulosa cells, it is unclear how cAMP mediates the contrasting effect of the gonadotropic hormones on granulosa cell growth and differentiation.

Numerous genes expressed in the ovary are regulated by the cAMP pathway as a consequence of gonadotropin signaling. During the rodent estrous cycle, FSH secreted from the anterior pituitary results in the expression of many FSH-responsive genes important for the growth and maturation of the ovarian follicle in the ovary. However in response to the preovulatory LH surge, many FSH-responsive genes are rapidly down-regulated.

The nuclear response to the cAMP pathway is mediated by a large family of transcription factors. The best characterized of these factors are the cAMP-Response Element (CRE)-Binding Protein (CREB), CRE Modulator (CREM) and the inducible isoforms of CREM. CREB and CREM genes encode several nuclear factors that can act as transcriptional activators or repressors of cAMP-responsive genes. These transcription factors exert their effects upon binding to CREs within the promoters of cAMP-responsive genes.

ICER is unique in that its expression is induced by cAMP from an internal promoter within the Crem gene. ICER shares the DNA binding and dimerization domains with the other CREM isoforms, but lacks the kinase and transactivation domains. ICER therefore functions as a dominant negative transcriptional repressor by binding as a homodimer or heterodimer with other CRE-binding family members. This feature endows ICER with a key role in mediating the repression of cAMP-dependent transcription. In the ovaries of adult cycling rats, Crem mRNA levels of ICER isoforms have been shown to be selectively induced in the granulosa cells of preovulatory follicles in response to the ovulatory surge of LH. Similarly, ICER expression was found to be induced in granulosa cells of PMSG-primed immature rats injected with human Chorionic Gonadotropin (hCG), whereas PMSG alone did not induce ICER expression. This induction of ICER in response to hCG has been proposed to mediate the suppression of FSH inducible genes, such as inhibin alpha (Inha) and cytochrome P450 (Cyp19a).

The anterior pituitary glycoprotein FSH is essential for folliculogenesis. Hypophysectomized rats lack sustained follicular growth due to the lack of gonadotropins. However, treatment with FSH promotes the formation of large antral follicles. Another major effect of FSH on granulosa cells is to induce the expression of LH receptors and acquire LH responsiveness. FSH has been shown to stimulate cyclin D2 mRNA via a cAMP/PKA pathway in granulosa cells. However, a luteinizing dose of LH in hormonally primed hypophysectomized female rats resulted in a rapid decrease in cyclin D2 mRNA and protein levels. Presently, it is unclear how cAMP mediates the actions of both FSH and LH in producing their contrasting effects on granulosa cell growth and cyclin D2 expression.

As disclosed in the example below, a mouse model with restrictive expression of ICER to the ovaries, particularly within the granulosa cells, was generated using a 2.5 kb fragment of the FSH/PMSG inducible inhibin alpha-subunit gene promoter. It was found that over-expression of ICER in the ovaries led to an increase in the ovulation rate either in the hormone-primed immature or mature cycling females. The finding was unexpected since CREM/ICER null mice did not display obvious female reproductive abnormalities (Blendy et al. Nature 1996; 380: 162-165 and Nantel et al. Nature 1996; 380: 159-162).

Inducible cAMP Early Repressor Polypeptides and Related Therapeutics

Inducible cAMP early repressor polypeptides are a group of polypeptides that bind to a CRE and suppress the expression of a gene under the control of the CRE, e.g., FSH-inducible gene. As disclosed herein, each of the polypeptides can have a sequence that is at least 75% (i.e., any percentage between 75% and 100% inclusive, e.g., 75%, 80%, 85%, 90%, 95%, 99%, and 100%) identical to any one of mouse/rat and human ICER I, Iγ, II, and IIγ, respectively. Listed below are their polypeptide sequences (SEQ ID NOs: 1-8) and nucleic acid sequences encoding them (SEQ ID NOs: 9-16).

1. Rat/mouse ICERI (isoform 6 of CREM):
(SEQ ID NO: 1)
MAVTGDETDEETDLAPSHMAAATGDMPTYQIRAPTTALPQGVVMAASPGS
LHSPQQLAEEATRKRELRLMKBREAAKECRRKKKEYVKCLENRVAVLENQ
NKTLIEELKALKDLYCHKAE
(SEQ ID NO: 9)
ATGGCTGTAACTGGAGATGAAACTGATGAGGAGACTGACCTTGCCCCAAG
TCACATGGCTGCTGCCACAGGTGACATGCCAACTTACCAGATCCGAGCTC
CTACTACTGCTTTGCCACAAGGTGTGGTGATGGCTGCCTCACCAGGAAGC
CTGCACAGTCCCCAGCAACTAGCAGAAGAAGCAACTCGCAAGCGGGAGCT
GAGGCTGATGAAAAACAGGGAAGCTGCCCGGGAGTGTCGCAGGAAGAAGA
AAGAATATGTCAAATGTCTTGAAAATCGTGTGGCTGTGCTTGAAAATCAA
AACAAGACCCTCATTGAGGAACTCAAGGCCCTCAAAGACCTTTATTGCCA
TAAAGCAGAG
2. Rat/mouse ICERIgamma (isoform 7 of CREM):
(SEQ ID NO: 2)
MAVTGDETAATGDMPTYQIRAPTTALPQGVVMAASPGSLHSPQQLAEEAT
RKRELRLMKBREAAKECRRKKKEYVKCLENRVAVLENQNKTLIEELKALK
DLYCHKAE
(SEQ ID NO: 10)
ATGGCTGTAACTGGAGATGAAACTGCTGCCACAGGTGACATGCCAACTTA
CCAGATCCGAGCTCCTACTACTGCTTTGCCACAAGGTGTGGTGATGGCTG
CCTCACCAGGAAGCCTGCACAGTCCCCAGCAACTAGCAGAAGAAGCAACT
CGCAAGCGGGAGCTGAGGCTGATGAAAAACAGGGAAGCTGCCCGGGAGTG
TCGCAGGAAGAAGAAAGAATATGTCAAATGTCTTGAAAATCGTGTGGCTG
TGCTTGAAAATCAAAACAAGACCCTCATTGAGGAACTCAAGGCCCTCAAA
GACCTTTATTGCCATAAAGCAGAG
3. Rat/mouse ICERII (isoform 8 of CREM):
(SEQ ID NO: 3)
MAVTGDETDEETDLAPSHMAAATGDMPTYQIRAPTTALPQGVVMAASPGS
LHSPQQLAEEATRKRELRLMKBREAAKECRRRKKEYVKCLESRVAVLEVQ
BKKLIEELETLKDICSPKTD
(SEQ ID NO: 11)
ATGGCTGTAACTGGAGATGAAACTGATGAGGAGACTGACCTTGCCCCAAG
TCACATGGCTGCTGCCACAGGTGACATGCCAACTTACCAGATCCGAGCTC
CTACTACTGCTTTGCCACAAGGTGTGGTGATGGCTGCCTCACCAGGAAGC
CTGCACAGTCCCCAGCAACTAGCAGAAGAAGCAACTCGCAAGCGGGAGCT
GAGGCTGATGAAAAACAGGGAAGCTGCTAAAGAATGTCGACGTCGAAAGA
AAGAGTATGTCAAGTGTCTTGAGAGTCGAGTCGCAGTGCTGGAAGTTCAG
AACAAGAAGCTTATAGAGGAGCTTGAAACTTTGAAAGACATTTGCTCTCC
CAAAACAGAT
4. Rat/mouse ICERIIgamma (isoform 9 of CREM):
(SEQ ID NO: 4)
MAVTGDETAATGDMPTYQIRAPTTALPQGVVMAASPGSLHSPQQLAEEAT
RKRELRLMKBREAAKECRRRKKEYVKCLESRVAVLEVQBKKLIEELETLK
DICSPKTD
(SEQ ID NO: 12)
ATGGCTGTAACTGGAGATGAAACTGCTGCCACAGGTGACATGCCAACTTA
CCAGATCCGAGCTCCTACTACTGCTTTGCCACAAGGTGTGGTGATGGCTG
CCTCACCAGGAAGCCTGCACAGTCCCCAGCAACTAGCAGAAGAAGCAACT
CGCAAGCGGGAGCTGAGGCTGATGAAAAACAGGGAAGCTGCTAAAGAATG
TCGACGTCGAAAGAAAGAGTATGTCAAGTGTCTTGAGAGTCGAGTCGCAG
TGCTGGAAGTTCAGAACAAGAAGCTTATAGAGGAGCTTGAAACTTTGAAA
GACATTTGCTCTCCCAAAACAGAT
5. Human ICER-I (sp|Q03060-8|CREM_HUMAN Isoform
6):
(SEQ ID NO: 5)
MAVTGDDTDEETELAPSHMAAATGDMPTYQIRAPTAALPQGVVMAASPGS
LHSPQQLAEEATRKRELRLMKNREAARECRRKKKEYVKCLENRVAVLENQ
NKTLIEELKALKDLYCHKVE
(SEQ ID NO: 13)
ATGGCTGTAACTGGAGATGAAACAGATGAGGAAACTGAACTTGCCCCAAG
TCACATGGCTGCTGCCACTGGTGACATGCCAACTTACCAGATCCGAGCTC
CTACTGCTGCTTTGCCACAGGGAGTGGTGATGGCTGCATCGCCCGGAAGT
TTGCACAGTCCCCAGCAGCTGGCAGAAGAAGCAACACGCAAACGAGAGCT
GAGGCTAATGAAAAACAGGGAAGCTGCCCGGGAGTGTCGCAGGAAGAAGA
AAGAATATGTCAAATGTCTTGAAAATCGTGTGGCTGTGCTTGAAAACCAA
AACAAGACTCTCATTGAGGAACTCAAGGCCCTCAGAGATCTTTATTGCCA
TAAAGTAGAGTAA
6. Human ICER-Igamma (sp|Q03060-9|CREM_HUMAN Iso-
form 7):
(SEQ ID NO: 6)
MAVTGDDTAATGDMPTYQIRAPTAALPQGVVMAASPGSLHSPQQLAEEAT
RKRELRLMKNREAARECRRKKKEYVKCLENRVAVLENQNKTLIEELKALK
DLYCHKVE
(SEQ ID NO: 14)
ATGGCTGTAACTGGAGATGAAACAGCTGCCACTGGTGACATGCCAACTTA
CCAGATCCGAGCTCCTACTGCTGCTTTGCCACAGGGAGTGGTGATGGCTG
CATCGCCCGGAAGTTTGCACAGTCCCCAGCAGCTGGCAGAAGAAGCAACA
CGCAAACGAGAGCTGAGGCTAATGAAAAACAGGGAAGCTGCCCGGGAGTG
TCGCAGGAAGAAGAAAGAATATGTCAAATGTCTTGAAAATCGTGTGGCTG
TGCTTGAAAACCAAAACAAGACTCTCATTGAGGAACTCAAGGCCCTCAGA
GATCTTTATTGCCATAAAGTAGAGTAA
7. Human ICER-II (sp|Q03060-10|CREM_HUMAN Isoform
8)
(SEQ ID NO: 7)
MAVTGDDTDEETELAPSHMAAATGDMPTYQIRAPTAALPQGVVMAASPGS
LHSPQQLAEEATRKRELRLMKNREAAKECRRRKKEYVKCLESRVAVLEVQ
NKKLIEELETLKDICSPKTDY
(SEQ ID NO: 15)
ATGGCTGTAACTGGAGATGAAACTGATGAGGAAACTGAACTTGCCCCAAG
TCACATGGCTGCTGCCACTGGTGACATGCCAACTTACCGGATCCGAGCTC
CTACTGCTGCTTTGCCACAGGGAGTGGTGATGGCTGCATCGCCCGGAAGT
TTGCACAGTCCCCAGCAGCTGGCAGAAGAAGCAACACGCAAACGAGAGCT
GAGGCTAATGAAAAACAGGGAAGCTGCCAAAGAATGTCGACGTCGAAAGA
AAGAATATGTAAAATGTCTGGAGAGCCGAGTTGCAGTGCTGGAAGTCCAG
AACAAGAAGCTTATAGAGGAACTTGAAACCTTGAAAGACATTTGCTCTCC
CAAAACAGATTAG
8. Human ICER-IIgamma (sp|Q03060-11|CREM_HUMAN
Isoform 9)
(SEQ ID NO: 8)
MAVTGDDTAATGDMPTYQIRAPTAALPQGVVMAASPGSLHSPQQLAEEAT
RKRELRLMKNREAAKECRRRKKEYVKCLESRVAVLEVQNKKLIEELETLK
DICSPKTDY
(SEQ ID NO: 16)
ATGGCTGTAACTGGAGATGAAACTGCTGCCACTGGTGACATGCCAACTTA
CCGGATCCGAGCTCCTACTGCTGCTTTGCCACAGGGAGTGGTGATGGCTG
CATCGCCCGGAAGTTTGCACAGTCCCCAGCAGCTGGCAGAAGAAGCAACA
CGCAAACGAGAGCTGAGGCTAATGAAAAACAGGGAAGCTGCCAAAGAATG
TCGACGTCGAAAGAAAGAATATGTAAAATGTCTGGAGAGCCGAGTTGCAG
TGCTGGAAGTCCAGAACAAGAAGCTTATAGAGGAACTTGAAACCTTGAAA
GACATTTGCTCTCCCAAAACAGATTAG

These ICER proteins all have the same functions as a transcriptional repressor. They are conserved between species with a high degree of sequence identity (>85%) between rodents, primates and other vertebrates. In humans, CREM/ICER is localized to chromosome band 10p11.2 and is present as a single copy per haploid genome. ICER inducibility by cAMP and autoregulation is conserved between species as well. The conservation of this function in humans further confirms the pivotal role of ICER as a nuclear target of the cAMP signal transduction pathway.

The percent identity of two amino acid sequences or of two nucleic acids is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

As disclosed herein, over-expression of ICER I, Iγ, II, or IIγ in an ovary can lead to a higher ovulation rate. Accordingly, the polypeptides, nucleic acids encoding them, and an agonist thereof can be used to increase ovulation in a vertebrate animal having an ovary. Conversely, antagonists of the polypeptides or nucleic acids can be used in decreasing ovulation.

An agonist of an ICER polypeptide is a compound that interacts with an ICER polypeptide to enhance its repressor activity. An antagonist of an ICER polypeptide is a compound that interferes with the repressor activity of the ICER polypeptide. Examples of the antagonist include, but are not limited to, proteins, peptides, small molecule compounds, RNAi agents, antisense nucleic acids, or antibodies.

While many ICER preparations can be used to practice this invention, a highly purified or isolated ICER polypeptide is one preferred embodiment. The terms “peptide,” “polypeptide,” and “protein” are used herein interchangeably to describe the arrangement of amino acid residues in a polymer. A peptide, polypeptide, or protein can be composed of the standard 20 naturally occurring amino acid, in addition to rare amino acids and synthetic amino acid analogs. They can be any chain of amino acids, regardless of length or post-translational modification (for example, glycosylation or phosphorylation). The peptide, polypeptide, or protein “of this invention” include recombinantly or synthetically produced fusion versions having the particular domains or portions that bind to a CRE and suppress the expression of a gene under the control of the CRE. The term also encompasses polypeptides that have an added amino-terminal methionine (useful for expression in prokaryotic cells).

An “isolated polypeptide” refers to a polypeptide that has been separated from other proteins, lipids, and nucleic acids with which it is naturally associated. The polypeptide can constitute at least 10% (i.e., any percentage between 10% and 100% inclusive, e.g., 20%, 30%, 40%, 50%, 60%, 70%, 80%, 85%, 90%, 95%, and 99%) by dry weight of the purified preparation. Purity can be measured by any appropriate standard method, for example, by column chromatography, polyacrylamide gel electrophoresis, or HPLC analysis. An isolated polypeptide of the invention can be purified from a natural source, produced by recombinant DNA techniques, or by chemical methods.

A “recombinant” peptide, polypeptide, or protein refers to a peptide, polypeptide, or protein produced by recombinant DNA techniques; i.e., produced from cells transformed by an exogenous DNA construct encoding the desired peptide. A “synthetic” peptide, polypeptide, or protein refers to a peptide, polypeptide, or protein prepared by chemical synthesis. The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified.

Within the scope of this invention are fusion proteins containing one or more of the afore-mentioned sequences and a heterologous sequence. A heterologous polypeptide, nucleic acid, or gene is one that originates from a foreign species, or, if from the same species, is substantially modified from its original form. Two fused domains or sequences are heterologous to each other if they are not adjacent to each other in a naturally occurring protein or nucleic acid.

The amino acid composition of the above-mentioned agonist or antagonist peptide/polypeptide/protein may vary without disrupting the ability to bind to a CRE and enhance or inhibit the respective cellular response. For example, it can contain one or more conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Thus, a predicted nonessential amino acid residue in one of SEQ ID NOs: 1-8 is preferably replaced with another amino acid residue from the same side chain family. Alternatively, mutations can be introduced randomly along all or part of the sequences, such as by saturation mutagenesis, and the resultant mutants can be screened for the ability to bind to the respective receptor and trigger the respective cellular response to identify mutants that retain the activity as descried below in the examples.

A functional equivalent of a peptide, polypeptide, or protein of this invention refers to a polypeptide derivative of the peptide, polypeptide, or protein, e.g., a protein having one or more point mutations, insertions, deletions, truncations, a fusion protein, or a combination thereof. It retains substantially the activity to of the above-mentioned agonist or antagonist peptides, polypeptides, or proteins. The isolated polypeptide can contain one of SEQ ID NOs: 1-8, or a functional fragment thereof. In general, the functional equivalent is at least 75% (e.g., any number between 75% and 100%, inclusive, e.g., 70%, 80%, 85%, 90%, 95%, and 99%) identical to one of SEQ ID NOs: 1-8.

A polypeptide described in this invention can be obtained as a recombinant polypeptide. To prepare a recombinant polypeptide, a nucleic acid encoding it can be linked to another nucleic acid encoding a fusion partner, e.g., glutathione-s-transferase (GST), 6x-His epitope tag, or M13 Gene 3 protein. The resultant fusion nucleic acid expresses in suitable host cells a fusion protein that can be isolated by methods known in the art. The isolated fusion protein can be further treated, e.g., by enzymatic digestion, to remove the fusion partner and obtain the recombinant polypeptide of this invention.

Alternatively, the peptides/polypeptides/proteins of the invention can be chemically synthesized (see e.g., Creighton, “Proteins: Structures and Molecular Principles,” W.H. Freeman & Co., NY, 1983), or produced by recombinant DNA technology as described herein. For additional guidance, skilled artisans may consult Ausubel et al. (supra), Sambrook et al. (“Molecular Cloning, A Laboratory Manual,” Cold Spring Harbor Press, Cold Spring Harbor, N.Y., 1989), and, particularly for examples of chemical synthesis Gait, M. J. Ed. (“Oligonucleotide Synthesis,” IRL Press, Oxford, 1984).

As an ICER functions as a transcription repressor, the above-disclosed therapeutic polypeptide can be associated with, e.g., conjugated or fused to, one or more of an amino acid sequence comprising a nuclear localization signal (NLS), a cell-penetrating peptide (CPP) sequence, a transcription repressor domain, and the like. In this manner, a composition of the invention as discussed below can include a transport enhancer. For example, the composition may include a penetration enhancing agent, such as MSM, for the delivery of the ICER or related therapeutic polypeptides to a cell and/or through the cell membrane and into the nucleus of the cell. The ICER or related therapeutic polypeptides then function to down-regulate transcription of a target gene, thereby resulting in an increase in ovulation. As indicated above, the ICER or related therapeutic polypeptides may be delivered by itself or as a fusion with one or more of an NLS, CPP, and/or other domains. See, e.g., Tachikawa et al. PNAS (2004) vol. 101, no. 42:15225-15230.

A cell-penetrating peptide (CPP) generally consists of less than 30 amino acids and has a net positive charge. CPPs internalize in living animal cells in vitro and in vivo in endocytotic or receptor/energy-independent manner. There are several classes of CPPs with various origins, from totally protein-derived CPPs via chimeric CPPs to completely synthetic CPPs. Examples of CPPs are known in the art. See, e.g., U.S. Application Nos. 20090099066 and 20100279918. It is know that CPPs can delivery an exogenous protein to ovary.

Although the ICER or related therapeutic polypeptides to be delivered may be fusion proteins including a NLS and/or CPP, in certain instances, the protein does not include an NLS and/or a CPP as the transport enhancer may serve the function of delivering the biologically active agent directly to the cell, and/or through the cell membrane into the cytoplasm of the cell and/or into the nucleus of the cell as desired. For instance, in certain instances, it may be desirable to deliver a biologically active protein to the cell wherein the protein is not conjugated or fused to another molecule. In such an instance, any biologically active protein may be delivered directly in conjunction with the transport enhancer.

All of naturally occurring ICER polypeptides, genetic engineered ICER polypeptides, and chemically synthesized ICER polypeptides can be used to practice the invention disclosed therein. ICER polypeptides obtained by recombinant DNA technology may have the same amino acid sequence as naturally a occurring ICER (one of SEQ ID NOs: 1-8) or an functionally equivalent thereof. They also include chemically modified versions. Examples of chemically modified ICER polypeptides include ICER polypeptides subjected to conformational change, addition or deletion of a side chain, and ICER polypeptides to which a compound such as polyethylene glycol has been bound. Once purified and tested by standard methods or according to the method described in the examples below, an ICER polypeptide can be included in pharmaceutical composition.

The present invention also provides a nucleic acid that encodes any of the polypeptides mentioned above. Preferably, the nucleotide sequences are isolated and/or purified. A nucleic acid refers to a DNA molecule (for example, but not limited to, a cDNA or genomic DNA), an RNA molecule (for example, but not limited to, an mRNA), or a DNA or RNA analog. A DNA or RNA analog can be synthesized from nucleotide analogs. The nucleic acid molecule can be single-stranded or double-stranded. An “isolated nucleic acid” is a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein.

The present invention also provides recombinant constructs having one or more of the nucleotide sequences described herein. Example of the constructs include a vector, such as a plasmid or viral vector, into which a nucleic acid sequence of the invention has been inserted, in a forward or reverse orientation. In a preferred embodiment, the construct further includes regulatory sequences, including a promoter, operably linked to the sequence. Large numbers of suitable vectors and promoters are known to those of skill in the art, and are commercially available. Appropriate cloning and expression vectors for use with prokaryotic and eukaryotic hosts are also described in Sambrook et al. (2001, Molecular Cloning: A Laboratory Manual, Cold Spring Harbor Press).

Examples of expression vectors include chromosomal, nonchromosomal and synthetic DNA sequences, e.g., derivatives of or Simian virus 40 (SV40), bacterial plasmids, phage DNA, baculovirus, yeast plasmids, vectors derived from combinations of plasmids and phage DNA, viral DNA such as vaccinia, adenovirus, fowl pox virus, and pseudorabies. However, any other vector may be used as long as it is replicable and viable in the host. The appropriate nucleic acid sequence may be inserted into the vector by a variety of procedures. In general, a nucleic acid sequence encoding one of the polypeptides described above can be inserted into an appropriate restriction endonuclease site(s) by procedures known in the art. Such procedures and related sub-cloning procedures are within the scope of those skilled in the art.

The nucleic acid sequence in the aforementioned expression vector is preferably operatively linked to an appropriate transcription control sequence (promoter) to direct mRNA synthesis. Examples of such promoters include: the retroviral long terminal (LTR) or SV40 promoter, the E. coli lac or trp promoter, the phage lambda PL promoter, and other promoters known to control expression of genes in prokaryotic or eukaryotic cells or viruses. In a preferred embodiment, the promoter is a tissue specific promoter that drives mRNA synthesis in an ovary. In a more preferred embodiment, the promoter is responsive to FSH, hCG, or PMSG. Examples include the 2.5 kb mouse inhibin alpha promoter mentioned in the example below and described in Hsu et al. Endocrinology 1995; 136: 5577-5586.

The expression vector can also contain a ribosome binding site for translation initiation, and a transcription terminator. The vector may include appropriate sequences for amplifying expression. In addition, the expression vector preferably contains one or more selectable marker genes to provide a phenotypic trait for selection of transformed host cells such as dihydrofolate reductase or neomycin resistance for eukaryotic cell cultures, or such as tetracycline or ampicillin resistance in E. coli.

The vector containing the appropriate nucleic acid sequences as described above, as well as an appropriate promoter or control sequence, can be employed to transform an appropriate host to permit the host to express the polypeptides described above (e.g., one of SEQ ID NOs: 1-8). Such vectors can be used in gene therapy. Examples of suitable expression hosts include bacterial cells (e.g., E. coli, Streptomyces, Salmonella typhimurium), fungal cells (yeast), insect cells (e.g., Drosophila and Spodoptera frugiperda (Sf9)), animal cells (e.g., CHO, COS, and HEK 293), adenoviruses, and plant cells. The selection of an appropriate host is within the scope of those skilled in the art. In some embodiments, the present invention provides methods for producing the above mentioned polypeptides by transfecting a host cell with an expression vector having a nucleotide sequence that encodes one of the polypeptides. The host cells are then cultured under a suitable condition, which allows for the expression of the polypeptide.

The present invention further provides gene therapy using nucleic acids encoding one or more of the polypeptides mentioned above or an analog or homolog thereof. Preferably, the gene therapy targets an ovary. Targeted gene therapy involves the use of vectors (e.g., organ-homing peptides) that are targeted to specific organs or tissues after systemic administration. For example, the vector can have a covalent conjugate of avidin and a monoclonal antibody to an ovary specific protein, such as a receptor expressed in granulosa cells.

In certain embodiments, the present invention provides gene therapy for the in vivo production of the above-mentioned ICER polypeptides. Such therapy would achieve its therapeutic effect by introduction of the nucleic acid sequences into cells or tissues of a human or a non-human animal in need of an increase in ovulation. Delivery of the nucleic acid sequences can be achieved using a recombinant expression vector such as a chimeric virus or a colloidal dispersion system. Preferred for therapeutic delivery of the nucleic acid sequences is the use of targeted liposomes.

Various viral vectors which can be utilized for gene therapy disclosed herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an ovary-specific antibody or hormone (e.g., LH or FSH) that has a receptor in an ovary cell (e.g., granulosa cell). Those of skill in the art will recognize that specific polynucleotide sequences can be inserted into the retroviral genome or attached to a viral envelope to allow target specific delivery of the retroviral vector.

Another targeted system for delivery of nucleic acids is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. RNA, DNA, and intact virions can be encapsulated within the aqueous interior and be delivered to cells in a biologically active form. Methods for efficient gene transfer using a liposome vehicle, are known in the art. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Exemplary phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

A nucleic acid sequence of this invention can be a DNA or a RNA. The terms “RNA,” “RNA molecule,” and “ribonucleic acid molecule” are used interchangeably herein, and refer to a polymer of ribonucleotides. The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA also can be chemically synthesized. DNA and RNA can be single-stranded (i.e., ssRNA and ssDNA, respectively) or multi-stranded (e.g., double-stranded, i.e., dsRNA and dsDNA, respectively).

A nucleic acid sequence can encode a small interference RNA (e.g., an RNAi agent) that targets one or more of genes encoding the above-mentioned ICER polypeptides and inhibits its expression or activity. The term “RNAi agent” refers to an RNA, or analog thereof, having sufficient sequence complementarity to a target RNA to direct RNA interference. Examples also include a DNA that can be used to make the RNA. RNA interference (RNAi) refers to a sequence-specific or selective process by which a target molecule (e.g., a target gene, protein or RNA) is down-regulated. Generally, an interfering RNA (“iRNA”) is a double stranded short-interfering RNA (siRNA), short hairpin RNA (shRNA), or single-stranded micro-RNA (miRNA) that results in catalytic degradation of specific mRNAs, and also can be used to lower or inhibit gene expression.

The term “short interfering RNA” or “siRNA” (also known as “small interfering RNAs”) refers to an RNA agent, preferably a double-stranded agent, of about 10-50 nucleotides in length, preferably between about 15-25 nucleotides in length, more preferably about 17, 18, 19, 20, 21, 22, 23, 24, or 25 nucleotides in length, the strands optionally having overhanging ends comprising, for example 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery (e.g., Dicer or a homolog thereof).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

The term “miRNA” or “microRNA” refers to an RNA agent, preferably a single-stranded agent, of about 10-50 nucleotides in length, preferably between about 15-25 nucleotides in length, more preferably about 17, 18, 19, 20, 21, 22, 23, 24, or 25 nucleotides in length, which is capable of directing or mediating RNA interference. Naturally-occurring miRNAs are generated from stem-loop precursor RNAs (i.e., pre-miRNAs) by Dicer. The term microRNA (or “miRNA”) is used interchangeably with the term “small temporal RNA” (or “stRNA”) based on the fact that naturally-occurring microRNAs (or “miRNAs”) have been found to be expressed in a temporal fashion (e.g., during development).

Thus, also within the scope of this invention is utilization of RNAi featuring degradation of RNA molecules (e.g., within a cell). Degradation is catalyzed by an enzymatic, RNA-induced silencing complex (RISC). A RNA agent having a sequence sufficiently complementary to a target RNA sequence (e.g., one or more of the above-mentioned genes) to direct RNAi means that the RNA agent has a homology of at least 50%, (e.g., 50%, 60%, 70%, 80%, 90%, 95%, 98%, 99%, or 100% homology) to the target RNA sequence so that the two are sufficiently complementary to each other to hybridize and trigger the destruction of the target RNA by the RNAi machinery (e.g., the RISC complex) or process. A RNA agent having a “sequence sufficiently complementary to a target RNA sequence to direct RNAi” also means that the RNA agent has a sequence sufficient to trigger the translational inhibition of the target RNA by the RNAi machinery or process. A RNA agent also can have a sequence sufficiently complementary to a target RNA encoded by the target DNA sequence such that the target DNA sequence is chromatically silenced. In other words, the RNA agent has a sequence sufficient to induce transcriptional gene silencing, e.g., to down-modulate gene expression at or near the target DNA sequence, e.g., by inducing chromatin structural changes at or near the target DNA sequence.

The above-mentioned polynucleotides can be delivered using polymeric, biodegradable microparticle or microcapsule delivery devices known in the art. Another way to achieve uptake of the polynucleotides is using liposomes, prepared by standard methods. The polynucleotide can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells (Cristiano, et al., 1995, J. Mol. Med. 73:479). Alternatively, tissue specific targeting can be achieved by the use of tissue-specific transcriptional regulatory elements that are known in the art. Delivery of naked DNA (i.e., without a delivery vehicle) to an intramuscular, intradermal, or subcutaneous site is another means to achieve in vivo expression.

siRNA, miRNA, and asRNA (antisense RNA) molecules can be designed by methods well known in the art. siRNA, miRNA, and asRNA molecules with homology sufficient to provide sequence specificity required to uniquely degrade any RNA can be designed using programs known in the art, including, but not limited to, those maintained on websites for AMBION, Inc. and DHARMACON, Inc. Systematic testing of several designed species for optimization of the siRNA, miRNA, and asRNA sequences can be routinely performed by those skilled in the art. Considerations when designing short interfering nucleic acid molecules include, but are not limited to, biophysical, thermodynamic, and structural considerations, base preferences at specific positions in the sense strand, and homology. These considerations are well known in the art and provide guidelines for designing the above-mentioned RNA molecules.

An antisense polynucleotide (preferably DNA) of the present invention can be any antisense polynucleotide so long as it possesses a base sequence complementary or substantially complementary to that of the DNA encoding an ICER polypeptide of this invention and capable of suppressing expression of the polypeptide. The base sequence can be at least about 70%, 80%, 90%, or 95% homology to the complement of the DNA encoding the polypeptide. These antisense DNAs can be synthesized using a DNA synthesizer.

The antisense DNA of the present invention may contain changed or modified sugars, bases or linkages. The antisense DNA may also be provided in a specialized form such as liposomes, microspheres, or may be applied to gene therapy, or may be provided in combination with attached moieties. Such attached moieties include polycations such as polylysine that act as charge neutralizers of the phosphate backbone, or hydrophobic moieties such as lipids (e.g., phospholipids, cholesterols, etc.) that enhance the interaction with cell membranes or increase uptake of the nucleic acid. Preferred examples of the lipids to be attached are cholesterols or derivatives thereof (e.g., cholesteryl chloroformate, cholic acid, etc.). These moieties may be attached to the nucleic acid at the 3′ or 5′ ends thereof and may also be attached thereto through a base, sugar, or intramolecular nucleoside linkage. Other moieties may be capping groups specifically placed at the 3′ or 5′ ends of the nucleic acid to prevent degradation by nucleases such as exonuclease, RNase, etc. Such capping groups include, but are not limited to, hydroxyl protecting groups known in the art, including glycols such as polyethylene glycol, tetraethylene glycol and the like. The inhibitory action of the antisense DNA can be examined using a cell-line or animal based gene expression system of the present invention in vivo and in vitro.

The above mentioned antagonist or agonist can be an antibody. In one example, the above mentioned antagonist is an antibody. The term “antibody” refers to an immunoglobulin molecule or immunologically active portion thereof, i.e., an antigen-binding portion. Examples include, but are not limited to, a protein having at least one or two, heavy (H) chain variable regions (V_{H}), and at least one or two light (L) chain variable regions (V_{L}). The V_{H} and V_{L} regions can be further subdivided into regions of hypervariability, termed “complementarity determining regions” (“CDR”), interspersed with regions that are more conserved, termed “framework regions” (FR). As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, and IgG4), delta, epsilon and mu constant region genes, as well as the myriad immunoglobulin variable region genes.

Antibodies that specifically bind to one of the above-mentioned ICER polypeptides can be made using methods known in the art. This antibody can be a polyclonal or a monoclonal antibody. Examples of such antibodies include those described in the working examples below. In one embodiment, the antibody can be recombinantly produced, e.g., produced by phage display or by combinatorial methods. In another embodiment, the antibody is a fully human antibody (e.g., an antibody made in a mouse which has been genetically engineered to produce an antibody from a human immunoglobulin sequence), a humanized antibody, or a non-human antibody, for example, but not limited to, a rodent (mouse or rat), goat, primate (for example, but not limited to, monkey), or camel antibody.

In another embodiment, the antagonist is a mutant form of the above-mentioned ICER polypeptide, which interferes with the above-mentioned pathway and therefore interferes with ICER's function. The term “mutant” encompasses naturally occurring mutants and mutants created chemically and/or using recombinant DNA techniques. A mutant of one of the above-mentioned wild type polypeptide can be due to alteration, e.g., truncation, elongation, substitution, deletion, or insertion, of one or more amino acids. The alteration also can have a modified amino acid, such as one comprising a post-translational modification.

### Compositions

This invention also provides a composition that contains a suitable carrier and one or more of the agents described above. The composition can be a pharmaceutical composition that contains a pharmaceutically acceptable carrier. The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate.

The above-described composition, in any of the forms described above, can be used for modulating ovulation. An effective amount refers to the amount of an active compound/agent that is required to confer a therapeutic effect on a treated subject. Effective doses will vary, as recognized by those skilled in the art, depending on the types of conditions treated, route of administration, excipient usage, and the possibility of co-usage with other therapeutic treatment.

A pharmaceutical composition of this invention can be administered parenterally, orally, nasally, rectally, topically, or buccally. The term “parenteral” as used herein refers to, but not limited to, subcutaneous, intracutaneous, intravenous, intrmuscular, intraarticular, or intraarterial injection, as well as any suitable infusion technique. A sterile injectable composition can be a solution or suspension in a non-toxic parenterally acceptable diluent or solvent. Such solutions include, but are not limited to, 1,3-butanediol, mannitol, water, Ringer's solution, and isotonic sodium chloride solution. In addition, fixed oils are conventionally employed as a solvent or suspending medium (e.g., synthetic mono- or diglycerides). Fatty acid, such as, but not limited to, oleic acid and its glyceride derivatives, are useful in the preparation of injectables, as are natural pharmaceutically acceptable oils, such as, but not limited to, olive oil or castor oil, polyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, TWEENS or SPANS or other similar emulsifying agents or bioavailability enhancers, which are commonly used in the manufacture of pharmaceutically acceptable solid, liquid, or other dosage forms also can be used for the purpose of formulation. As used herein, “administering” does not include microinjection of a fertilized oocyte and intergenerational transmission via germ cells.

### Uses

As described herein, the aforementioned ICER polypeptides can be used to enhance, stimulate, promote or otherwise increase ovulation. As such, any ICER peptide/protein that has an activity that is similar to the activity of a peptide of any one of SEQ ID NOs: 1-8 may be used in the treatment of infertility disorders. The infertility disorders that may be treated include any disorder that may benefit from an increase in the amount of ova. Preferably, the increase in ova results in an appreciable increase in pregnancy. The ICER polypeptide/nucleic acid-based compositions of the invention may be used in any protocol suitable for treatment of a fertility disorder. As such, the therapeutics of the present invention may be used in the treatment of anovulatory females in the induction of ovulation and pregnancy in anovulatory infertile patients in whom the cause of infertility is functional and not due to primary ovarian failure or individuals suffering from hypogonadotropic hypogonadism.

The protocols for the administration of the ICER polypeptides may be similar to the protocols for the administration of other biologics. For example to stimulate ovulation, the protein-based compositions (e.g., a polypeptide of amino acid sequence of any one of SEQ ID NOs: 1-8) may be prescribed for five days each cycle, typically as a single daily dose on each specified day. However, the dosage may be increased or decreased by a physician based on the patient's individual response. In a typical treatment, it may be necessary to perform an ICER polypeptide load test determine whether the patient will be responsive to the polypeptide in the manner shown e.g., U.S. Pat. No. 5,091,170.

In one example, the ICER polypeptide can be initially administered beginning on cycle day 3 and taken daily until cycle day 7. At cycle day 9 or 10, the LH and FSH levels of the patient can be monitored using ovulation predictor kits. If a surge has not occurred by cycle day 16, an ultrasound may be performed to check for follicular development and measure the thickness of the uterine lining. After LH surge ovulation should occur with two days. If pregnancy does not result, a further cycle of ICER therapeutic can be administered again. In such a subsequent cycle, at the onset of menstrual flow, before day three, a pelvic examination and or ultrasound check may be performed.

In another example, where the above protocol is unsuccessful in producing a pregnancy, intrauterine insemination may be used to improve possibility of conception. Such intrauterine insemination may be combined with one or more of clomiphene, letrozole, HMG/insemination or Gonal-F/Follistim injections and intrauterine insemination. In intrauterine insemination, a baseline ultrasound can be performed on or before cycle day 3. Beginning at day 3 the ICER therapy can be initiated and continued to cycle day 7. In combined therapies, the patient may be treated with the ICER polypeptide or related nucleic acids, in combination with clomiphene (or letrozole)/FSH or HMG/and intrauterine insemination. In such protocols, an injection of 150 units of FSH or HMG may be administered on day 8 or 9. On cycle 9 or 10 LH and FSH are determined.

The ICER polypeptide or related nucleic acids may be combined with other agents or treatments for infertility to produce ovulation stimulation. Such additional treatments include administration of other stimulators of gonadotropin release e.g., clomiphene and letrozole, as well as various gonadotropins to increase ovulation induction and/or follicle maturation.

For example, exogenous FSH may be provided in a course of daily administrations lasting between 7 to 12 days. Numerous FSH preparations are commercially available and may be used in the methods of the invention. Such commercial preparations include urinary-derived FSH compositions and recombinant FSH compositions, such as Pergonal™ and Fertinex™, (Serono Laboratories Inc., Randolph, Mass.), Repronex™ (Ferring Pharmaceutical Inc., Tarrytown, N.J.), Humegomm™ (Organon, West Orange, N.J.), and Follistim™. In addition to FSH, other gonadotropin hormones can be used in the methods and related kits described herein. Such hormones include hCG, which is commercially available as Novarel (Ferring Pharmaceutical Inc., Tarrytown, N.J.) and Pregnyl™ (Organon, West Orange, N.J.).

The pharmaceutical compositions and treatment methods of the invention are useful in fields of human medicine and veterinary medicine in connection with in vitro fertilisation (IVF) or other assisted reproductive technology (ART). Thus the subject to be treated may be a vertebrate animal, preferably human or domestic animals (including livestock animal, laboratory test animals, and companion animals). For veterinary purposes, subjects include for example, farm animals, companion animals, exotic and/or zoo animals, laboratory animals including, and poultry.

The term “animal” refers to all animals including primates (e.g. humans, monkeys), livestock animals (e.g. sheep, cows, horses, donkeys, goats, pigs), laboratory test animals (e.g. mice, rats, guinea pigs, rabbits, hamsters), companion animals (e.g. dogs, cats), captive wild animals (e.g. emus, kangaroos, deer, foxes, tigers, pandas), ayes (e.g. chickens, bantams, ducks, emus, geese, pheasants, ostriches, and turkeys), reptiles (e.g. lizards, snakes, frogs) and fish (e.g. trout, salmon). The term “livestock animal” refers to a domesticated animal raised in an agricultural setting to produce commodities such as food (e.g., meat, eggs, and milk), fiber, and labor or in a sport/race setting to produce profit. Examples include cattle, donkeys, dogs, horses, goats, sheep, pigs, camels, deer, poultry, and farmed fish. Animals of endangered species refer to animals that are at risk of becoming extinct because they are either few in numbers, or threatened by changing environmental or predation parameters. Examples of animals of endangered species include, but are not limited to, those identified by the International Union for Conservation of Nature and Natural Resources (also known as the IUCN Red List) or U.S. Fish and Wildlife Service Endangered Species Program (see ecos.fws.gov/tess_public/pub/listedAnimals.jsp), such as Hawaiian crow, Wyoming toad, Spix's macaw, Socorro dove, red-tailed black shark, scimitar oryx, Catarina pupfish, mountain gorilla, Bactrian camel, Ethiopian wolf, saiga, takhi, iberian lynx, kakapo, Arakan forest turtle, Sumatran rhinoceros, Javan rhino, Brazilian merganser, axolotl, leatherback sea turtle, northern white rhinoceros, gharial, vaquita, Philippine eagle, brown spider monkey, California condor, island fox, black rhinoceros, Chinese alligator, Sumatran orangutan, Asiatic cheetah, African wild ass, Hawaiian monk seal, red wolf, blue whale, Asian elephant, giant panda, and bald eagle.

The present invention also contemplated kits for use in the treatment of fertility disorders. Such kits include at least a first composition containing the proteins/peptides described above in a pharmaceutically acceptable carrier. The kits may additionally contain solutions or buffers for affecting the delivery of the first composition. The kits may further contain additional compositions which contain further stimulators of FSH production/release e.g., additional other ICER derived proteins, other stimulators, e.g., clomiphene and/or further hormones such as e.g., hCG, LH and the like. The kits may further contain catheters, syringes or other delivering devices for the delivery of one or more of the compositions used in the methods of the invention. The kits may further contain instructions containing administration protocols for the therapeutic regimens.

### Example

### Materials and Methods

Animals

FVB mice (Taconic Farms, Germantown, N.Y.) were used in the present studies. Animals were housed and handled in accordance with protocols approved by the Institutional Animal Care and Use Committee (IACUC) of the University of Medicine and Dentistry of New Jersey.

Generation of Transgene Cassettes

The mouse ICER-IIγ cDNA (SEQ ID NO: 12) was subcloned into the pCMV2-FLAG to generate an amino terminal tagged ICER IIγ expression plasmid. The 5′ end of the FLAG-ICER DNA was extended with the 45 bp region of the 5′ UTR of ICER fused to the ATG of FLAG by two rounds of PCR using the following primers: Forward primer 1^{st} Round (5′-CCT GCA GTG GAC TGT GGT ACG GCC AAT AAG ACC ACT CTA TAT GC-3′, SEQ ID NO: 17), Forward primer 2^{nd} Round with PstI site (5′-ACC ACT CTA TAT GCA AAA GCC CAA CAT GGA CTC CAA AGA C-3′, SEQ ID NO: 18) with Reverse primer containing an XbaI site (5′-CGT CTA GAT ACT AAT CTG TTT TGG GAG-3′, SEQ ID NO: 19). The final PCR product was subcloned into PCR 2.1 TOPO vector. The SV40 intron and ploy A site was PCR amplified from pGL2-Basic using the following primers: Forward (5′-CCG TCT AGA AAT GTA ACT GTA TTC AGC G-3′, SEQ ID NO: 20) and Reverse (5′-CGC TGC AGC TCG AGA GAC ATG ATA AGA TAC ATT G-3′, SEQ ID NO: 21) and subcloned into PCR 2.1 TOPO vector. The UTR-FLAG-ICER IIγ DNA was excised from TOPO vector as an XbaI/XbaI fragment and inserted in the XbaI site upstream of the SV40 intron poly (A) signal of the SV40 TOPO plasmid. The resulting construct was then digested with PstI to cut out the 1.4 kb fragment. The 3′ over-hang ends were blunt ended with T4 DNA polymerase and subsequently subcloned into the EcoRV site of Bluescript (SK−) downstream of the 2.5 kb mouse inhibin alpha promoter (Hsu et al. Endocrinology 1995; 136: 5577-5586). For the production of Tg mice, the transgene cassettes were isolated by excising the XhoI fragment from the resulting Bluescript (SK−) final construct and was microinjected into fertilized oocytes of FVB×FVB mice. The injected oocytes were then implanted into pseudopregnant CD-1 females (Charles River).

Genotyping

Genomic DNA was extracted from tail biopsies obtained from 2 to 3 week old mice using the phenol:chloroform method as described by The Jackson Laboratory. Genomic DNA was digested for 4 hr with BamHI and XbaI. After digestion, DNA was fractionated by electrophoresis in 0.8% agarose gel, denatured and transferred to a nylon membrane (Hybond-N+, GE HEALTHCARE). Radiolabeled DNA fragment comprising the FLAG-ICER region of the transgene was used as hybridization probe. Hybridization was done overnight at 65° C. in hybridization solution (5×SSC, 5×Denhardt's reagent, 0.5% SDS) including 200 μg/ml denatured salmon sperm DNA. The membrane was washed twice with 2×SSC, 0.1% SDS at 65° C. for 30 min and twice with 1×SSC, 0.1% SDS at 65° C. for 30 min. Membranes were exposed to films for autoradiography.

RNase Protection Assay

RNA was extracted from primary granulosa cells or ovarian tissues using TRIZOL Reagent (INVITROGEN) according to the manufacture's instructions. Aliquots of 5 μg of total RNA were subjected to RNase protection analysis. Briefly, RNase Protection assays were performed using the RPA III Ribonuclease Protection Assay kit (AMBION) following the manufacturer's instructions using radioactive riboprobes that recognizes either the exogenous or endogenous ICER transcripts. A mouse β-actin probe was used for loading control where indicated. Samples were separated on a denaturing polyacrylamide gel (6% polyacrylamide/8 M urea) and the gels were vacuum-dried at 80° C. and visualized by autoradiography.

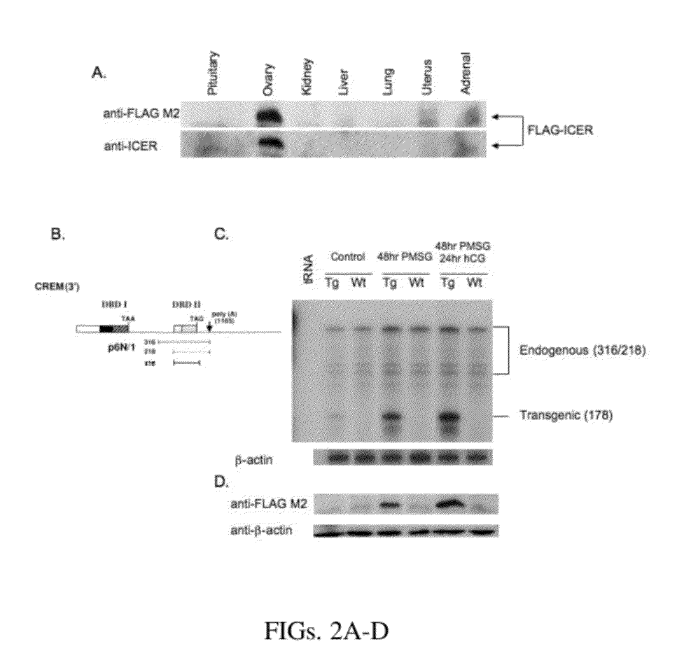

As shown in FIG. 2B, under the CREM gene are the predicted protected fragments produced using the probe (p6N/1) (Foulkes et al. Cell 1991; 64: 739-749) in RNase protection assays. Since FLAG-ICER consists of the cDNA of ICER-IIγ it would only produce a protected fragment of 178 bp. Endogenous CREM isoforms will produce protected fragments of 316 and 218 bps.

[α-³²P] UTP Labeling of RNA Probe

The p6N/1 template DNA used to detect exogenous ICER and the p75 template DNA used to detect endogenous ICER have been previously described (Laoide et al. EMBO J 1993; 12: 1179-1191). For loading control, pTRI-β-actin-mouse antisense control templates (AMBION, Austin, Tex.) were used. The RNA probes used in the RNase protection assay were generated using Maxiscript In vitro Transcription Kit (AMBION) following the manufacturer's instructions. The probes were purified and free nucleotides were removed by column purification using PROBE QUANT G-50 Micro-column.

Immunoblotting

Whole cell protein lysates (obtained using TRIZOL reagent) were boiled and protein quantification was performed using BIO-RAD DC reagent with BSA as a standard. Equal amounts of protein (20-30 μg) were mixed with 2× loading buffer (20% glycerol, 0.1% bromophenol blue, 2 mM DTT). Samples were boiled for 5 min and loaded onto 15% SDS-polyacrylamide gels. The gels were run at 200 volts for 55 min. The proteins were transferred onto PVDF membrane for 80 min at 100 volts. The membranes were blocked for 10 min in 1×PBS containing 5% non-fat milk or BSA (Following the recommendations of the manufacturer of the antibody being used) and probed overnight at 4° C. with either anti-FLAG M2-peroxidase (HRP) monoclonal antibody (SIGMA 1:500) or with a previously characterized rabbit polyclonal anti-ICER antibody (1:1000). Blots were washed in 1×PBS with 0.1% TWEEN-20 four times for 15 min each, then probed with anti-rabbit horseradish peroxidase conjugated antibody at a 1:20,000 dilution in 5% non-fat milk for 45 min, washed 4 times for 4 min each in 1×PBS with 0.1% TWEEN-20 and visualized using enhanced chemiluminescence and autoradiography.

Immunohistochemistry

Ovaries were fixed in 10% formalin for 24 hr, dehydrated, embedded in paraffin and sectioned at 5 p.m. Standard hematoxylin and eosin staining was also preformed. Briefly, sections were baked in an oven at 56° C. for 30 min, followed by deparafinization in Xylene. Sections were then rehydrated in a series of sequential ethanol baths ranging from 100%, 95%, 75% and 50%. Sections were rinsed and subjected to peroxidase quenching. An antigen retrieval step was preformed using 0.01M Citrate Buffer solution (pH 8.0). Sections were either incubated with a 1:5,000 dilution of a rabbit polyclonal antibody that recognizes ICER or a 1:2,500 dilution of a mouse monoclonal antibody that recognizes FLAG overnight at 4° C. Sections were then washed and incubated with a biotinylated goat anti-rabbit antibody or biotinylated goat anti-mouse antibody (ZYMED LABORATORIES Inc.) for 10 min at room temperature. Sections were then washed, incubated for 10 min at room temperature with a horseradish peroxidase-conjugated streptavidin (ZYMED LABORATORIES Inc.), washed, and incubated with a Subrate-Chromogen Mixture (ZYMED LABORATORIES Inc.) for 10 min at room temperature for antigen detection (brown reaction product). Sections were counterstained with hematoxylin and analyzed by light microscopy.

Gonadotropin Treatment and Oocyte Harvesting

Intraperitoneal (IP) Gonadotropin administrations were performed as described in Sicinski et al. Nature 1996; 384: 470-474. Mice were subsequently sacrificed at various time points after PMSG or hCG treatment and the ovaries were removed and either immediately frozen in liquid nitrogen and stored at −80° C. until further processed or immediately fixed in 10% formalin.

Immature FVB females (3-4 weeks) were superovulated by IP injection with 5 IU of PMSG (at noon) followed 48 h later with 5 IU hCG and mated with FVB stud males. The next morning, upon detection of a vaginal plug, the fertilized oocytes were collected from the oviduct in M2 medium. The embryos were then incubated in M2 with hyalurinadase (300 ug/ml) to detach the follicle cells from the embryos, washed several time with M2 media and incubated in M16 medium at 37° C. in 5% CO₂. The fertilized oocytes were cultured for 4 days and allowed to develop to the blastocyst stage. After 4 days the total number of blastocysts were determined and a percentage from the total number of embryo were calculated.

Determination of oocyte release in mature, 2-month old, wild-type or transgenic mice was preformed by introducing a stud male. Female mice were checked for the presence of a vaginal plug every morning as indirect indication of ovulation, subsequently sacrificed and fertilized embryos collected as described above.

Quantitative Determination of Hormones

Progesterone levels in superovulated immature transgenic or wild-type mice were measured from serum using blood collected by cardiac puncturing. Serum progesterone levels were assessed using a direct solid-phase enzyme-immunoassay (DRG Progesterone ELISA kit) according to the manufacturer's instructions. Before the assay, extraction of steroids from serum was preformed using 6.6 vol of ethyl ether; the extracts were dried and reconstituted in zero standard control serum provided by the manufacturer. The reconstituted extracts from transgenic and wild-type mice were assayed in duplicates. Progesterone concentration measured by a spectrophotometer at 450 nm, against a standard curve constructed using a four parameter logistic function.

Statistical Analysis

Differences between treatments were analyzed for significance by Student's t-test.

### Results

Generation of an Ovarian Specific Transgenic of ICER

Numerous genes expressed in the ovary are regulated by the cAMP pathway as a consequence of gonadotropin signaling. During the rodent estrous cycle, FSH secreted from the anterior pituitary results in the expression of many FSH-responsive genes important for the growth and maturation of the ovarian follicle in the ovary. However in response to the preovulatory LH surge, many FSH-responsive genes are rapidly down-regulated. ICER has been implicated in the transcriptional repression of FSH inducible genes during folliculogenesis. Yet no in vivo model systems to date are available to further elucidate ICER role in ovarian function. Therefore, ovarian specific ICER transgenic mice were generated.

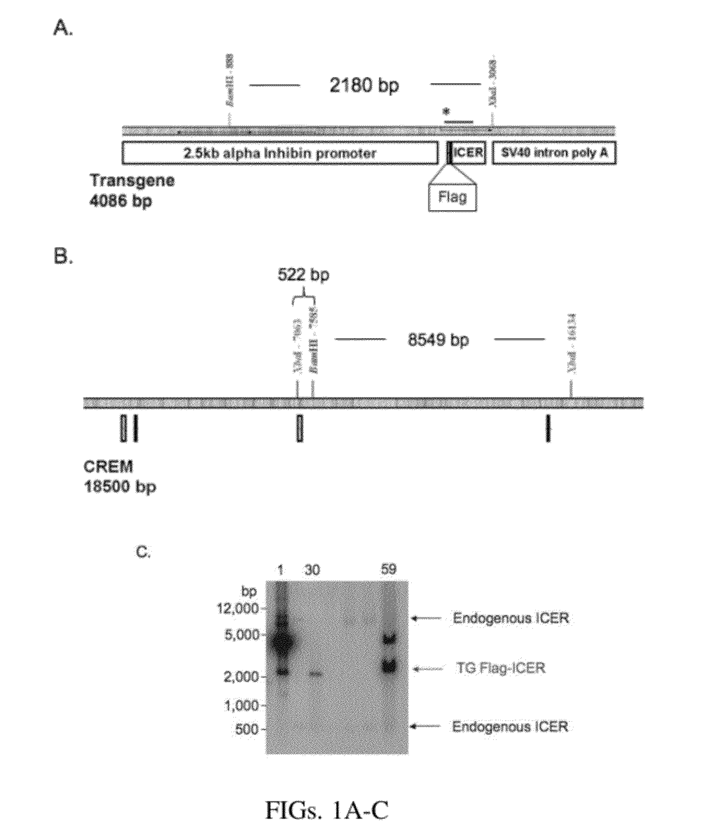

The DNA construct used to generate the transgenic mice contained FLAG-ICER cDNA sequence, flanked by 48 base pairs derived from the 5′ UTR of the endogenous ICER RNA, under the control of a 2.5 kb fragment of the FSH/PMSG inducible inhibin alpha-subunit gene promoter (FIG. 1A) to restrict the expression of the transgene to the granulosa cell compartment of the ovarian follicle as previously demonstrated (Hsu et al. Endocrinology 1995; 136: 5577-5586). After the DNA construct microinjection into fertilized oocytes from FVB mouse strain three founders were identified by Southern blot. Genotyping was performed using genomic DNA extracted from mouse-tail biopsies of 2-week-old pups. The extracted DNA was analyzed by Southern blot using a probe against FLAG-ICER to detect the 2.18 kb BamHI-XhoI fragment generated from the integrated cassette. The probe would also detect three endogenous CREM gene fragments, at 522 bp, 8.5 kb and 18.5 kb from the resulting digestion (FIG. 1B). Three independent transgenic founder mice TGN1, TGN30 and TGN59 were identified (FIG. 1C). At eight weeks of age, the three transgenic founder mice were backcrossed to FVB female mice. Two of the lines (TGN1 and TGN59) transmitted the transgene to the F1 generation and were phenotypically indistinguishable from each other and from their wild-type littermates.

Characterization of Transgenic Mice Lines

Ovarian specific expression of FLAG-ICER was confirmed by Western blot analysis using lysates prepared from different tissues of mature TGN1 mice. FIG. 2A shows that FLAG-tagged ICER was specifically expressed in the ovary when protein lysates were probed with antibodies raised against ICER or those specific for FLAG peptide. Similar results were obtained with TGN59. In order to assess the ovarian responsiveness of the transgenic mice to exogenous gonadotropin treatment, immature 3-week old mice were injected with either PMSG alone for 48 hr or followed by hCG for 24 hr post PMSG. RNA and protein levels of exogenous ICER were induced in the ovaries of transgenic mice TGN1 48 hr after PSMG treatment (FIGS. 2 C-D). Enhanced expression of the transgene is detected in the transgenic mice 24 hr after hCG treatment when compared to PMSG treatment alone. Similar results were also obtained with TGN59.

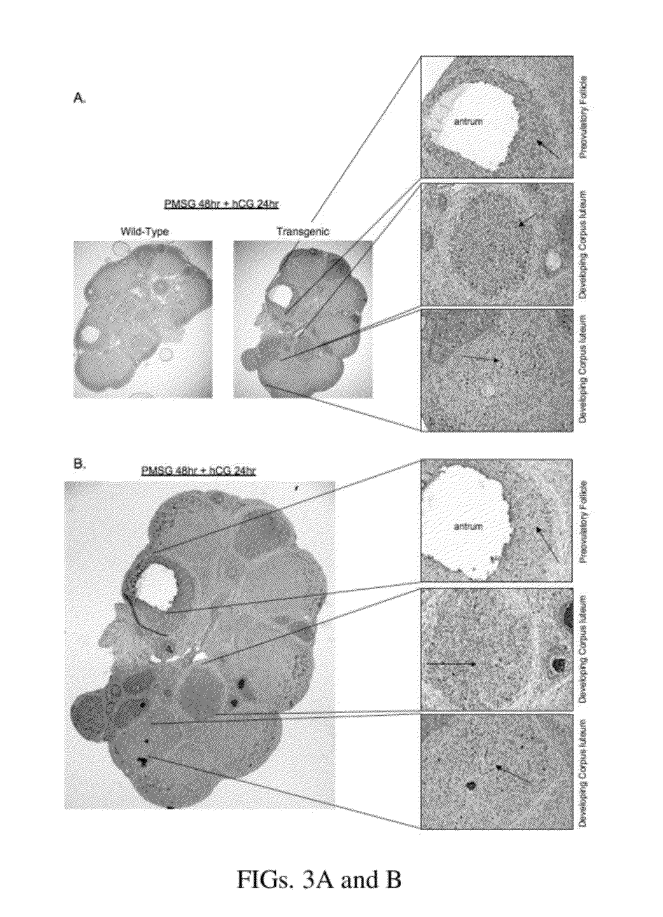

The localization of the induced transgene expression within the ovarian compartment was determined by immunohistochemical analysis. PMSG-primed immature female mice were injected with hCG and their ovaries were collected 24 hr later and sectioned for analysis. It was observed that exogenous gonadotropin treatment resulted in tissue specific expression of FLAG-ICER within the granulosa cells of the preovulatory follicle and in the luteinizing granulosa cells of the developing corpus luteum in tissues probed either with anti-ICER or anti-FLAG M2 antibody (FIGS. 3A-B). These results were consistent in both transgenic lines. This data collectively demonstrates successful generation of an animal model where the expression of ICER is induced with PMSG prior to hCG stimulation in granulosa cells. Furthermore, an enhanced expression beyond 12 hr of hCG treatment was achieved. This mouse model clearly manifests alterations in the temporal expression pattern of ICER in the ovaries, contrasting with the normally occurred expression of endogenous ICER in response to exogenous gonadotropins (Mukherjee et al. Mol Endocrinol 1998; 12: 785-800).

Phenotypic Characterization of the Ovarian Specific ICER Transgenic Mice

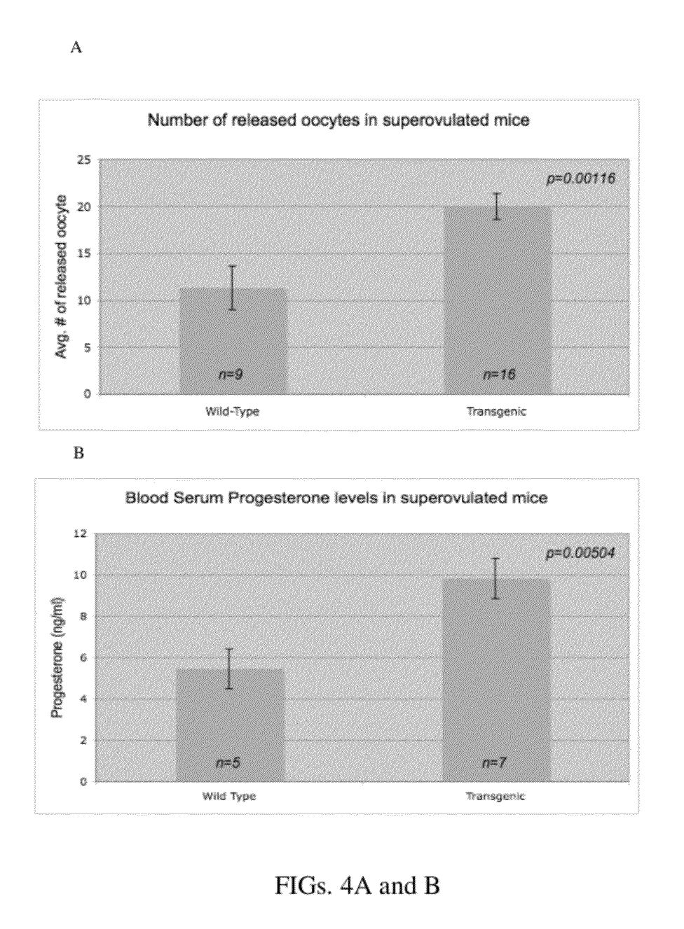

In order to determine the physiological effect of FLAG-ICER over-expression on ovarian function, in particular on ovulation, assays were carried out to compare the numbers of released oocytes between immature transgenic and wild type mice. Flag-tagged ICER transgenic mice displayed hypersensitivity to the ovulatory effects of PMSG and hCG, resulting in a two-fold increase in released oocytes compared to wild type littermates (FIG. 4A and Table 1).

TABLE 1
In vitro culture of oocytes

| | Control | Transgenic |
|---|---|---|
| Number of oocytes | 53* | 144* |
| | (n = 5)† | (n = 7)† |
| Blastocysts¶ (%) | 83 | 82.6 |

Sexually immature, control (wild-type) and Flag ICER transgenic female were superovulated and mated with wild-type males.
*Total number of oocytes isolated and used for in vitro culture
†Number of females from which oocytes were isolated.
¶Percentage of fertilized oocytes that formed blastocysts.

However, an increase in litter size between transgenic and wild-type female mice was not observed. This apparent discrepancy is likely related to the probability that in superovulation experiments, a limited number of blastocysts implantation could be a determining factor. In order to rule out intrinsic abnormalities due to ICER over-expression or as a result of hyperovulation, oocyte viability was assessed. The harvested oocytes were subjected to in vitro culture and maturation. The percentage of oocytes proceeding to blastocysts as seen after 4 days in culture were found to be comparable between transgenic and wild type mice (Table 1), suggesting that implantation rather then maturation would account the lack of increase litter size in the transgenic female mice.

In light of the above observation, assays were carried out to measure serum progesterone levels to determine whether the increased number of released oocytes correlates with an increased number of developing corpus luteum. It was found that transgenic mice exhibited twice the serum progesterone level as wild type mice following superovulation (FIG. 4B). This observation complements and supports the two-fold rate increase in ovulation observed in superovulated transgenic mice.

Ovulation Rate During the Estrous Cycle in Transgenic Mice

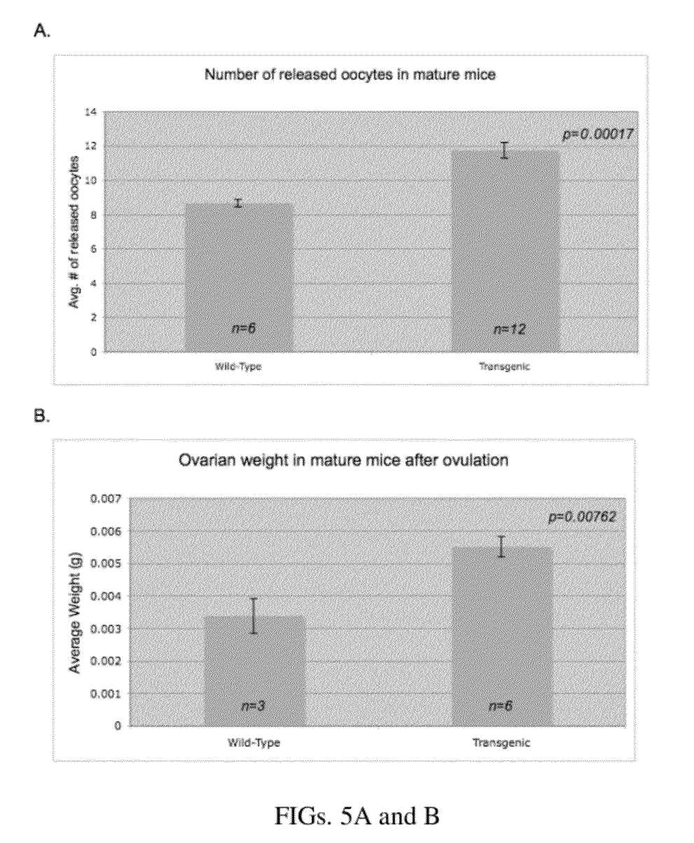

Since there was an increase rate of released oocytes in gonadotropin stimulated immature mice, experiments were carried out determine if mature transgenic mice release more oocytes then wild-type mice. In this experiment, two-month old female mice were mated with wild-type male FVB mice and sacrificed upon the detection of a vaginal plug, during which time the ovaries and oocytes were collected. As shown in FIG. 5A, mature transgenic females ovulated (11.75±0.46; n=12) significantly more than wild-type littermates (8.67±0.21; n=6).

To determine if the observed rate of ovulation correlates with changes in ovarian weight due to the consequent rise in corpora lutea formation, ovarian weights were assessed. It was found that transgenic ovaries weighed (5.51±0.31 mg; n=6) significantly more than wild-type (3.38±0.53 mg; n=3) (FIG. 5B). Collectively, increased rate of ovulation in these animals correlated with an increase in the number of CL in the ovaries, which was reflected by an increase in ovarian weight.

Hormonal Profile in Transgenic Mice During the Estrous Cycle

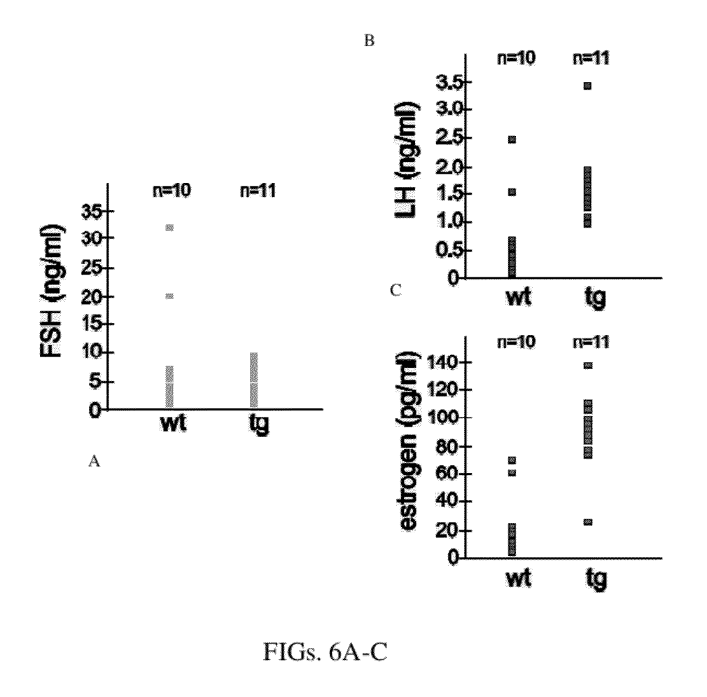

The increase in ovarian weight observed in adult females due to increase in CL found in the ovaries would suggest an increase in hormonal production. In order to determine the hormonal levels, blood sera collected from three-month old estrus cycling transgenic and wild-type mice were obtained for hormonal analysis. No differences were detected in FSH serum levels. However there was a detectable increase in LH levels of in the transgenic mice. Serum estradiol levels were also found to be elevated in transgenic mice (FIG. 6). These results clearly indicate that the hyperovulation observed in the transgenic mice was accompanied by an imbalance in hormonal level.

Crem-mutant mice were generated resulting in male sterility due to postmeiotic arrest at the first step of spermatogenesis, whereas the female mice were reported to be fertile (Blendy et al. Nature 1996; 380: 162-165 and Nantel et al. Nature 1996; 380: 159-162). However detailed studies of the reproductive processes which occur in the female have not yet been reported. Since the homozygous mutant mice lacked activators and repressors such as CREM and ICER, it was suspected that the balance mechanism controlling CREM-mediated gene expression in Crem-null mice might be affected in two opposite directions, which would result in a mutual cancellation without apparent phenotypic differences in female mice. ICER and CREM proteins are both detected in the ovaries hence, the above-described ovarian-specific ICER transgenic animals can aid in answering these questions by disrupting this presumed balance.

As disclosed herein, exogenous levels of FLAG-ICER transcript and protein were highly detected in the ovaries of the transgenic mice when subjected to gonadotropin treatment. Immunohistochemistry analysis of the ovaries of the transgenic mice treated with PMSG and hCG showed that the spatial expression of FLAG-ICER is similar to the endogenous expression mainly localized to the granulosa cells of antral follicles as well as in the luteal cells of the developing corpus luteum. However, the temporal pattern of expression of exogenous ICER in the transgenic mice strongly deviated from the previously reported expression pattern of endogenous ICER. Ovaries from immature rats subjected to exogenous gonadotropin treatment normally express ICER within 4 hr of hCG treatment and declines after 7 hr, whereas immature transgenic female mice display induced levels of exogenous ICER 48 hr after of PMSG treatment with enhanced and sustained expression with the subsequent treatment of hCG for 24 hrs. Moreover in mature, estrous cycling, transgenic mice the temporal pattern of expression of the transgene displayed a biphasic expression during diestrus and proestrus, contrast to observed expression pattern of endogenous ICER, which is normally expressed only during proestrus. Surprisingly, these transgenic mice display biphasic surge in LH, during proesterus and in late estrus/early mettestrus. The consequence in the altered temporal expression of ICER may account for the altered levels of LH detected in the transgenic mice.

It was also shown that the selective expression of ICER in the ovaries of transgenic mice results in enhanced ovulation rate. The finding is particularly surprising since CREM/ICER null mice, as mentioned above, did not display obvious female reproductive abnormalities (Blendy et al. Nature 1996; 380: 162-165 and Nantel et al. Nature 1996; 380: 159-162). Perhaps by specifically altering the expression of one of the CREM isoforms, ICER, the selective expression of ICER probably negated any potential equilibrium that may have existed between the transcriptional activators and repressors in the ovaries.

Nevertheless, the results here strongly suggest that ICER regulates ovulation. It is hypothesized that ICER may inhibit the degeneration and resorption of the ovarian follicle before it reaches maturity and ovulates, a process referred to as follicular atresia. Inhibiting follicular atresia would rescue atretic follicles from their destined fate, resulting in the increase the number of follicles that mature and ovulate. Another possibility is that the follicles become more receptive to the effects of gonadotropins, which would lead to more efficient ovulation events.

Since ICER has previously been implicated in the transcriptional repression of two FSH responsive genes, Inha and Cyp19a1, the subsequent alteration in the temporal expression of ICER in the transgenic mice prior to an ovulatory cue, could essentially mimic the phenotype manifested in mice with null mutations in Inha or Cyp19a1. However, Inha null mice display high levels of serum FSH and the peptide hormone activin, which enhances FSH synthesis and secrection, ultimately resulting in infertility in female mice before succumbing to granulosa cell tumors (Matzuk et al. Nature 1992; 360: 313-319 and Matzuk et al. Proc Natl Acad Sci USA 1994; 91: 8817-8821). Furthermore, female mice with null mutations on Cyp19a1 manifest an increase in circulating testosterone levels with subsequent alterations in FSH and LH levels, resulting in infertility due to disruption in folliculogenesis (Britt et al. J Steroid Biochem Mol Biol 2001; 79: 181-185). Whereas null mutations in cyclin D2 has been shown to lead to infertility due to lack of granulosa cell proliferation resulting in the disruption of folliculogenesis. Consequently, it had been assumed that ICER transgenic animals would be infertile based on previous data showing ICER regulation of cyclin D2).

However, the ICER transgenic mice model surprisingly displayed enhanced ovulatory rates in response to stimulatory effects of the gonadotropins. To date, this is the first transgenic mouse model of its kind. As surprising as the findings described here might be, it is important to state that the above-described adult transgenic mice did not express the transgene throughout the entire length of the estrous cycle, nor did they display a gross over-expression of ICER levels.

The 2.5 kb portion of the Inha gene contains a CRE which has been proposed as the site of transcriptional activation by CREB in response to FSH, followed by the transcription repression during the LH surge as a result of ICER induction. In the transgenic animals described herein, FLAG-ICER inducibility in mature mice may be followed by an auto-regulation of the transgene, accounting for the lack of sustained transgene expression, until the preovulatory LH surge that result in the expression of the transgene again. Although theoretically the Inha gene is normally repressed as a result of the LH surge, the 2.4 kb region used here may not contain the regulatory elements required for LH repression. This effect was more drastic in superovulated mice where hCG treatment in PMSG primed mice resulted in a prolonged expression of the transgene. It is possible that FLAG-ICER mediate gene regulation at certain stages of the estrous cycle with alternating relief of the regulation upon auto-regulating its own expression. This intricate expression system may promote and enhance response to the ovulatory cues thereby resulting in hyperovulation.

The above-described transgenic mice can be used in investigating the differences in gene regulation between transgenic and wild-type mice prior to ovulation so as to identify a mechanism by which these animals become hyper-sensitive to ovulatory cues. These animals provide a suitable model to perform in vivo microarray analysis, which would allow one to unravel the underlying genes regulation responsible for the observed hyperovulation. Consequently, these transgenic mice constitute a unique model to study fertility and the physiological cues that would enable one to control the rate of ovulation.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated herein in their entireties.

What is claimed is:

1. A method of increasing ovulation in a vertebrate animal having an ovary, comprising administering to said animal a first composition comprising (i) an inducible cAMP early repressor polypeptide or (ii) a vector comprising a nucleic acid sequence encoding said polypeptide.

2. The method of claim 1, further comprising administering to the animal a second composition that stimulates ovulation.

3. The method of claim 2, wherein the second composition comprises a hormonal or a chemical stimulant of ovulation.

4. The method of claim 3, wherein the hormonal stimulant is a gonadotropin hormone selected from the group consisting of pregnant mare serum gonadotropin (PMSG), human menopausal gonadotropin (hMG), follicle stimulating hormone (FSH), luteinizing hormone (LH), human chorionic gonadotropin (hCG), and anti-Müllerian hormone (AMH).

5. The method of claim 1, wherein the animal is a mammal.

6. The method of claim 5, wherein the mammal is a human.

7. The method of claim 6, wherein the human has an infertility disorder.

8. The method of claim 7, wherein the infertility disorder is polycystic ovary syndrome (PCOS) or oligomenorrhea.

9. The method of claim 1, wherein the animal is a fish or a bird.

10. The method of claim 1, wherein the animal is a domestic animal.

11. The method of claim 10, wherein the domestic animal is a livestock animal.

12. The method of claim 10, wherein the domestic animal is a pet animal.

13. The method of claim 1, wherein the animal is an animal of an endangered species.

14. The method of claim 1, wherein the first composition is administered to said ovary of said animal.

15. The method of claim 1, wherein the inducible cAMP early repressor polypeptide comprises the sequence of any one of SEQ ID NOs: 1-8.

16. The method of claim 1, wherein the first composition is a pharmaceutical composition.

17. The method of claim 1, further comprising collecting one or more ova from said animal.

18. A kit for treating infertility, comprising
a first composition comprising (i) an inducible cAMP early repressor polypeptide or (ii) a vector comprising a nucleic acid sequence encoding said polypeptide, and
a second composition for treating infertility.

19. The kit of claim 18, wherein the second composition comprises a hormonal or a chemical stimulant of ovulation.

20. The kit of claim 19, wherein the hormonal stimulant is a gonadotropin hormone selected from the group consisting of pregnant mare serum gonadotropin (PMSG), human menopausal gonadotropin (hMG), follicle stimulating hormone (FSH), luteinizing hormone (LH), human chorionic gonadotropin (hCG), and anti-Müllerian hormone (AMH).

21. A method of decreasing ovulation in a vertebrate animal comprising administering to the animal a composition comprising an antagonist of an inducible cAMP early repressor polypeptide.

22. The method of claim 21, wherein the antagonist is a protein, a peptide, a small molecule compound, an antisense nucleic acid, or an RNAi agent.

23. The method of claim 1, wherein the inducible cAMP early repressor polypeptide is a mutant or polypeptide analog derived from the sequence of any one of SEQ ID NOs: 1-8.


SEQUENCE LISTING (21 sequences)

SEQ ID NO: 1 - protein, 120 aa, Mus musculus
MAVTGDETDEETDLAPSHMAAATGDMPTYQIRAPTTALPQGVVMAASPGSLHSPQQLAEEATRKRELRLMKBREAAKECRRKKKEYVKCLENRVAVLENQNKTLIEELKALKDLYCHKAE

SEQ ID NO: 2 - protein, 108 aa, Mus musculus
MAVTGDETAATGDMPTYQIRAPTTALPQGVVMAASPGSLHSPQQLAEEATRKRELRLMKBREAAKECRRKKKEYVKCLENRVAVLENQNKTLIEELKALKDLYCHKAE

SEQ ID NO: 3 - protein, 120 aa, Mus musculus
MAVTGDETDEETDLAPSHMAAATGDMPTYQIRAPTTALPQGVVMAASPGSLHSPQQLAEEATRKRELRLMKBREAAKECRRRKKEYVKCLESRVAVLEVQBKKLIEELETLKDICSPKTD

SEQ ID NO: 4 - protein, 108 aa, Mus musculus
MAVTGDETAATGDMPTYQIRAPTTALPQGVVMAASPGSLHSPQQLAEEATRKRELRLMKBREAAKECRRRKKEYVKCLESRVAVLEVQBKKLIEELETLKDICSPKTD

SEQ ID NO: 5 - protein, 120 aa, Homo sapiens
MAVTGDDTDEETELAPSHMAAATGDMPTYQIRAPTAALPQGVVMAASPGSLHSPQQLAEEATRKRELRLMKNREAARECRRKKKEYVKCLENRVAVLENQNKTLIEELKALKDLYCHKVE

SEQ ID NO: 6 - protein, 108 aa, Homo sapiens
MAVTGDDTAATGDMPTYQIRAPTAALPQGVVMAASPGSLHSPQQLAEEATRKRELRLMKNREAARECRRKKKEYVKCLENRVAVLENQNKTLIEELKALKDLYCHKVE

SEQ ID NO: 7 - protein, 121 aa, Homo sapiens
MAVTGDDTDEETELAPSHMAAATGDMPTYQIRAPTAALPQGVVMAASPGSLHSPQQLAEEATRKRELRLMKNREAAKECRRRKKEYVKCLESRVAVLEVQNKKLIEELETLKDICSPKTDY

SEQ ID NO: 8 - protein, 109 aa, Homo sapiens
MAVTGDDTAATGDMPTYQIRAPTAALPQGVVMAASPGSLHSPQQLAEEATRKRELRLMKNREAAKECRRRKKEYVKCLESRVAVLEVQNKKLIEELETLKDICSPKTDY

SEQ ID NO: 9 - DNA, 360 bp, Mus musculus
atggctgtaactggagatgaaactgatgaggagactgaccttgccccaagtcacatggctgctgccacaggtgacatgccaacttaccagatccgagctcctactactgctttgccacaaggtgtggtgatggctgcctcaccaggaagcctgcacagtccccagcaactagcagaagaagcaactcgcaagcgggagctgaggctgatgaaaaacagggaagctgcccgggagtgtcgcaggaagaagaaagaatatgtcaaatgtcttgaaaatcgtgtggctgtgcttgaaaatcaaaacaagaccctcattgaggaactcaaggccctcaaagacctttattgccataaagcagag

SEQ ID NO: 10 - DNA, 324 bp, Mus musculus
atggctgtaactggagatgaaactgctgccacaggtgacatgccaacttaccagatccgagctcctactactgctttgccacaaggtgtggtgatggctgcctcaccaggaagcctgcacagtccccagcaactagcagaagaagcaactcgcaagcgggagctgaggctgatgaaaaacagggaagctgcccgggagtgtcgcaggaagaagaaagaatatgtcaaatgtcttgaaaatcgtgtggctgtgcttgaaaatcaaaacaagaccctcattgaggaactcaaggccctcaaagacctttattgccataaagcagag

SEQ ID NO: 11 - DNA, 360 bp, Mus musculus
atggctgtaactggagatgaaactgatgaggagactgaccttgccccaagtcacatggctgctgccacaggtgacatgccaacttaccagatccgagctcctactactgctttgccacaaggtgtggtgatggctgcctcaccaggaagcctgcacagtccccagcaactagcagaagaagcaactcgcaagcgggagctgaggctgatgaaaaacagggaagctgctaaagaatgtcgacgtcgaaagaaagagtatgtcaagtgtcttgagagtcgagtcgcagtgctggaagttcagaacaagaagcttatagaggagcttgaaactttgaaagacatttgctctcccaaaacagat

SEQ ID NO: 12 - DNA, 324 bp, Mus musculus
atggctgtaactggagatgaaactgctgccacaggtgacatgccaacttaccagatccgagctcctactactgctttgccacaaggtgtggtgatggctgcctcaccaggaagcctgcacagtccccagcaactagcagaagaagcaactcgcaagcgggagctgaggctgatgaaaaacagggaagctgctaaagaatgtcgacgtcgaaagaaagagtatgtcaagtgtcttgagagtcgagtcgcagtgctggaagttcagaacaagaagcttatagaggagcttgaaactttgaaagacatttgctctcccaaaacagat

SEQ ID NO: 13 - DNA, 363 bp, Homo sapiens
atggctgtaactggagatgaaacagatgaggaaactgaacttgccccaagtcacatggctgctgccactggtgacatgccaacttaccagatccgagctcctactgctgctttgccacagggagtggtgatggctgcatcgcccggaagtttgcacagtccccagcagctggcagaagaagcaacacgcaaacgagagctgaggctaatgaaaaacagggaagctgcccgggagtgtcgcaggaagaagaaagaatatgtcaaatgtcttgaaaatcgtgtggctgtgcttgaaaaccaaaacaagactctcattgaggaactcaaggccctcagagatctttattgccataaagtagagtaa

SEQ ID NO: 14 - DNA, 327 bp, Homo sapiens
atggctgtaactggagatgaaacagctgccactggtgacatgccaacttaccagatccgagctcctactgctgctttgccacagggagtggtgatggctgcatcgcccggaagtttgcacagtccccagcagctggcagaagaagcaacacgcaaacgagagctgaggctaatgaaaaacagggaagctgcccgggagtgtcgcaggaagaagaaagaatatgtcaaatgtcttgaaaatcgtgtggctgtgcttgaaaaccaaaacaagactctcattgaggaactcaaggccctcagagatctttattgccataaagtagagtaa

SEQ ID NO: 15 - DNA, 363 bp, Homo sapiens
atggctgtaactggagatgaaactgatgaggaaactgaacttgccccaagtcacatggctgctgccactggtgacatgccaacttaccggatccgagctcctactgctgctttgccacagggagtggtgatggctgcatcgcccggaagtttgcacagtccccagcagctggcagaagaagcaacacgcaaacgagagctgaggctaatgaaaaacagggaagctgccaaagaatgtcgacgtcgaaagaaagaatatgtaaaatgtctggagagccgagttgcagtgctggaagtccagaacaagaagcttatagaggaacttgaaaccttgaaagacatttgctctcccaaaacagattag

SEQ ID NO: 16 - DNA, 327 bp, Homo sapiens
atggctgtaactggagatgaaactgctgccactggtgacatgccaacttaccggatccgagctcctactgctgctttgccacagggagtggtgatggctgcatcgcccggaagtttgcacagtccccagcagctggcagaagaagcaacacgcaaacgagagctgaggctaatgaaaaacagggaagctgccaaagaatgtcgacgtcgaaagaaagaatatgtaaaatgtctggagagccgagttgcagtgctggaagtccagaacaagaagcttatagaggaacttgaaaccttgaaagacatttgctctcccaaaacagattag

SEQ ID NO: 17 - DNA, 44 bp, Artificial
  Synthetic primer
cctgcagtggactgtggtacggccaataagaccactctatatgc

SEQ ID NO: 18 - DNA, 39 bp, Artificial
  Synthetic primer with Pstl site
accactctatatgcaaaagcccaacatggactccaaaga

SEQ ID NO: 19 - DNA, 27 bp, Artificial
  Synthetic primer with Xbal site
cgtctagatactaatctgttttgggag

SEQ ID NO: 20 - DNA, 28 bp, Artificial
  Synthetic primer
ccgtctagaaatgtaactgtattcagcg

SEQ ID NO: 21 - DNA, 34 bp, Artificial
  Synthetic primer
cgctgcagctcgagagacatgataagatacattg